(19) United States
(12) Patent Application Publication
(10) Pub. No.: US 2003/0050334 A1
(43) Pub. Date: Mar. 13, 2003

(54) Process for extraction of Delta-9-Tetrahydrocannabinol and other related cannabinoids and preparation of specific strength marijuana cigarettes

(76) Inventors: Ram B. Murty, Lexington, KY (US); Dipak K. Chowdhury, Lexington, KY (US); Murty Mangena, Lexington, KY (US)

Correspondence Address: LAW OFFICES OF TOWNSEND & BANTA, Suite 500, #50028, 1225 Eye Street, N.W., Washington, DC, 20005, US

(21) Appl. No.: 10/134,520

(22) Filed: Apr. 30, 2002

Related U.S. Application Data
(60) Provisional application No. 60/286,968, filed on Apr. 30, 2001.

(51) Int. Cl. C07D 311/80; A61K 31/353

(52) U.S. Cl. 514/454; 549/390

(57) ABSTRACT

A process for supercritical fluid extraction of delta-9-tetrahydrocannabinol (delta-9-THC), delta-8-THC, cannabinoids or other medicinal value compounds from marijuana and other plants. Preferably, the extraction is carried out with a solvent of liquid carbon dioxide alone, or in combination with a solvent of ethanol, methanol, isopropanol, and other nonpolar/semipolar solvents at a temperature and pressure to maintain the solvents in a supercritical state. The extraction process is preferably carried out for a period of from 0 to 9 hours. The extraction process conditions result in different strengths of extracted marijuana and selective isolation of extracted compounds or mixtures of compounds. The processed marijuana leaves or other parts of the marijuana plant can be used in the manufacture of different strengths of cigarettes for the delivery of delta-9-THC or other related compounds, or as adjuvant drugs for antiinflammatory and analgesic treatment, especially for chronic and terminal pain, neuropathic pain symptoms in humans, and in animals. Further, spiking methods can be used to make cigarettes of different strengths containing delta-9-THC or other related compounds, either synthetic or natural. Placebo cigarettes can also be prepared with pharmacologically negligible quantities of an active compound. The isolated compounds, or mixture of isolated compounds and adjuvants, of the extracted compounds can be used for the treatment of the above mentioned symptoms, either through cigarettes or by other suitable delivery systems.

### FIELD OF THE INVENTION

The present invention relates in general to the isolation/extraction from plant materials of pharmacologically active ingredients therein, and more particularly, to the extraction from marijuana plant parts of Delta-9-Tetrahydrocannabinol (THC) and other related compounds using one or more supercritical fluids. The present invention also provides a method of preparation of cigarettes (a drug delivery device) having differing specific concentrations of ingredients from the extracted marijuana leaves and other parts with the aid of spiking with either synthetic or natural compounds or mixture of compounds. In addition, placebo cigarettes can be prepared using the present method, having negligible quantities of Delta-9-THC therein. The isolated active compound or mixture of compounds can be used in different delivery devices for the treatment of pain.

### BACKGROUND OF THE INVENTION

Marijuana plants have been used since antiquity for herbal medicine and intoxication. Marijuana has been reported as having more than 30 different medical uses such as treating pain, nausea and vomiting associated with chemotherapy, wasting syndrome and appetite stimulation for AIDS patients, glaucoma, and neurological symptoms including muscle spasticity.

During the past twenty years there has been a steady increase in the illicit use of opiates. Among the opiates, Cannabis sativa (marijuana) or parts thereof, the major pharmacologically active component of which is Δ⁹-tetrahydrocannabinol (Δ⁹-THC), continues to be the most frequently abused drug, especially among young adults and school children. As a result, concerns regarding the pathophysiology of marijuana on the human organ system have been investigated.

The primary route of administration of marijuana (Δ⁹-THC) is via smoking thereof. Marijuana smoking has been the topic of a number of clinical and basic research studies, which have focused on the mechanism of the addictive processes and the health hazards associated with marijuana use. One of the major drawbacks in these studies has been the unavailability of placebo marijuana cigarettes depleted of Δ⁹-THC (i.e., a control), and research marijuana cigarettes containing standardized amounts of Δ⁹-THC. These studies have been further complicated by a lack of quantitative information on the effective delivery of Δ⁹-THC resulting from the varied and unpredictable amount of Δ⁹-THC usually found in marijuana cigarettes.

Consistency of the content of Δ⁹-THC in marijuana cigarettes is desirable because it overcomes the natural variation of concentration of Δ⁹-THC present in the marijuana due to latitude, weather, and soil conditions. Moreover, drug product consistency is a basic tenet of pharmacology and toxicology, since it enables standardized dosing for regulatory and treatment purpose. Also, when interpreting studies purporting to show the harmful effects of smoked marijuana, cannabinoid effects cannot be separated from the effects of inhaling smoke from burning plant material and contaminants. In addition, placebo cigarettes are desired, as they may be used as control cigarettes in investigations to determine the psychological and biological effects, as well as health hazards, associated with marijuana smoking.

The current debate on medical use of marijuana over the health risk began nation wide. Several states passed ballot initiatives in support of medical marijuana. At the present time Δ⁹-THC, the primary active ingredient in marijuana is an FDA-approved drug marketed as MARINOL capsules. A study recently published by the Institute of Medicine recommended that as a rapid-onset delivery system, smoked marijuana may be given on a short-term basis to patients with debilitating symptoms (such as intractable pain or vomiting). It is also recommended that smoked marijuana may be administered as a first step towards the possible development of alternative cannabinoid delivery systems. Currently, MARINOL (sold as capsules in 2.5 mg, 5 mg and 10 mg strength) is the only cannabinoid with approval for marketing in the United States. Thus, different Δ⁹-THC strength cigarettes spiked from placebo marijuana would be valuable for marijuana researchers and patients.

A prerequisite for the manufacture of placebo cigarettes is the standardized decannabinized marijuana i.e., marijuana containing pharmacologically insignificant levels of Δ⁹-THC. Procedures for decannabinization of marijuana are needed that will not affect the color, texture and physical properties of the marijuana plant material, and yet be suitable for cigarette manufacturing.

The last 20 years have seen an intense interest in the use of supercritical fluids in separation science. Supercritical fluid extraction is defined as the use of supercritical fluids to selectively remove analytes from solid, semisolid and liquid matrices. A supercritical fluid exhibits gas-like mass transfer properties and liquid-like solubility properties, enabling it to carry out solvent extractions much more efficiently and rapidly than a solvent in the liquid state. The significant properties of supercritical fluids that relate to extraction processes are: (a) solvating power directly related to density, (b) relatively high diffusivity and low viscosity, and (c) minimal surface tension.

The limitations, concerns, and restrictions associated with conventional methods of extraction can easily be overcome by using supercritical fluid (SCF) extraction. Supercritical Fluid Extraction Systems (SCFE) have been used for selective extraction of valuable chemicals from various natural, as well as synthetic, matrices under environmentally safe operation. The composite device used for this extraction technique has several components, i.e., a high-pressure pump, extraction vessel, back pressure regulator, and analyte collection vessel, besides the source of solvent (e.g., CO₂).

Most current commercial applications of SCF extractions involve biologically produced materials. This SCF technique is particularly relevant to extraction of biological compounds in cases where there is a requirement for low temperature processing, high mass transfer rates and negligible carry over of solvent into the final product. A comparison of SCFE and Soxhlet extraction of several compounds (e.g. polychlorinated biphenyls) has been made, and it has been found that supercritical fluid extraction time is shorter than the extraction times associated with conventional methods.

In the past few decades, experimental efforts have concentrated on utilizing SCFE techniques for applications such as (a) extraction of aroma producing compounds from fruits and coffee, flavors from foods, eugenol from clove buds, lanolin from wool, nicotine from tobacco, (b) production of spice extracts with a natural composition, (c) production of caffeine-free coffee, and (d) isolation of specialty chemicals.

Essential oils from medicinal plants have been extracted by SCF. It was observed that the ester constituents of the extracted material were high because the possibility of hydrolysis is reduced. An optimization procedure for the SCF extraction of cocaine has also been investigated using a near critical mixture of CO₂ and polar modifiers to extract major alkaloids from poppy straw. The extraction of thebaine, codeine, and morphine has been achieved by percolating a mixture of carbon dioxide-methanol-water (70:24:6 w/w, respectively) at 45° C. and 200 bar through a column containing poppy straw (previously ground and sieved) for 20 minutes.

Other compounds that have been extracted using SCF extraction techniques include steroids, trichothecenes, and ouabin (a steroid derived glycoside with eight hydroxyl groups) using 100% CO₂ under various pressures at 40° C. The application of SCF extraction for direct extraction of active ingredients from a liquid pharmaceutical matrix has been described in the extraction of sulfamethoxazole and trimethoprim from SEPTRA infusion. The extraction was carried out to determine whether SCF extraction could be used to remove the polar drug from the polar matrix. Hydrocarbon and typically lipophillic compounds of relatively low polarity, e.g., esters, lactones and epoxides, can be extracted in the low pressure range (i.e. 70-100 bar), but strongly polar substances (sugars, amino acids) need higher pressures for extraction.

### SUMMARY OF INVENTION

In order to overcome the deficiencies of the conventional methods discussed above, and to provide a marijuana cigarette having a consistent concentration of Δ⁹-THC and method of making same, a process is provided for the decannabinization of marijuana using supercritical fluid (SCF) extraction. In such process of the present invention, chromatographic methods (HPLC/GC) can be used to determine the amounts of Δ⁹-THC and other compounds in the marijuana and cigarettes.

In a first embodiment of the present invention, a process for the extraction of Delta-9-THC and other related pharmaceutically active compounds from marijuana plants is provided comprising super critical fluid extraction of Delta-9-THC, Delta-8-THC and related cannabinoids from marijuana plants using liquid carbon dioxide as a supercritical fluid.

In a second embodiment of the present invention according to the first embodiment above, the process further comprises the use of an organic cosolvent modifier in the extraction of Delta-9-THC, Delta-8-THC and other related pharmaceutically active compounds therein, from marijuana plants.

In a third embodiment of the present invention according to the first and second embodiments above, the supercritical fluids used in the supercritical fluid extraction process are one or more selected from the group comprising NH₃, N₂O, ethanol, pentane, and propane, and high purity carbon dioxides.

In a fourth embodiment of the present invention according to the first through third embodiments above, the super critical fluid extraction using CO₂ is preferably carried out at or above its critical temperature of 31.3° C. and at a pressure of 70 bar.

In a fifth embodiment according to the first through fourth embodiments above, the super critical fluid extraction process is carried out within an operating temperature range of from 31 to 120° C.

In a sixth embodiment of the present invention according to the first through fifth embodiments above, the supercritical fluid extraction process is carried out within an operating temperature range of from about 25-65° C.

In a seventh embodiment of the present invention according to the first through fifth embodiments above, the supercritical fluid extraction process is carried out within an operating temperature range of from about 30-65° C.

In an eighth embodiment of the present invention according to the first through fifth embodiments above, the supercritical fluid extraction process is carried out within an operating temperature range of from about 35-45° C.

In a ninth embodiment according to the first through eighth embodiments above, the supercritical fluid extraction process is carried out within a preferred pressure range of from about 70 to about 680 bar.

In a tenth embodiment of the present invention according to the first through eighth embodiments above, the supercritical fluid extraction process is carried out within a preferred pressure range of from about 100 to about 500 bar.

In an eleventh embodiment of the present invention according to the first through eighth embodiments above, the supercritical fluid extraction process is carried out within a preferred pressure range of from about 400 to about 500 bar.

In a twelfth embodiment of the present invention according to the first through eleventh embodiments above, the supercritical fluid extraction process is carried out within a time period of from 0 to 24 hours.

In a thirteenth embodiment of the present invention according to the first through eleventh embodiments above, the supercritical fluid extraction process is carried out within a time period of from 2 to 15 hours.

In a fourteenth embodiment of the present invention according to the first through eleventh embodiments above, the supercritical fluid extraction process is carried out within a time period of from 3 to 9 hours.

In a fifteenth embodiment according to the first through fourteenth embodiments above, the super critical fluid extraction process is carried out using a combination of carbon dioxide and an organic cosolvent modifier.

In an sixteenth embodiment of the present invention according to the fifteenth embodiment above, the organic cosolvent modifier is one or more selected from the group consisting of ethanol, methanol, 2-propanol, diethylether, ethyl acetate, chloroform, carbontetrachloride, acetonitrile, cyclohexane, acetone, acetic acid, nitromethane, dioxane, methylene chloride, hexane, pentane, acetylene, and pyridine.

In a seventeenth embodiment of the present invention, a supercritical fluid extraction process for extracting Delta-9-THC, Delta-8-THC and related pharmaceutically active compounds is provided, wherein the supercritical fluids used in said process comprise one or more selected from the group consisting of carbon dioxide, carbon monoxide, water, ethane, ammonia, nitrous oxide, fluoroform, and xenon.

In an eighteenth embodiment of the present invention according to the seventeenth embodiment above, the super critical fluid extraction process is carried out within an operating temperature range of from 31 to 120 ° C.

In a nineteenth embodiment of the present invention according to the seventeenth embodiment above, the supercritical fluid extraction process is carried out within an operating temperature range of from about 25-65° C.

In a twentieth embodiment of the present invention according to the seventeenth embodiment above, the supercritical fluid extraction process is carried out within an operating temperature range of from about 30-65° C.

In an twenty first embodiment of the present invention according to the seventeenth through twentieth embodiments above, the supercritical fluid extraction process is carried out within an operating temperature range of from about 35-45° C.

In a twenty second embodiment of the present invention according to the seventeenth through twenty first embodiments above, the supercritical fluid extraction process is carried out within a preferred pressure range of from about 70 to about 680 bar.

In a twenty third embodiment of the present invention according to the seventeenth through twenty first embodiments above, the supercritical fluid extraction process is carried out within a preferred pressure range of from about 100 to about 500 bar.

In a twenty fourth embodiment of the present invention according to the seventeenth through twenty first embodiments above, the supercritical fluid extraction process is carried out within a preferred pressure range of from about 400 to about 500 bar.

In a twenty fifth embodiment according to the seventeenth through twenty fourth embodiments above, the supercritical fluid extraction process is carried out within a time period of from 0 to 24 hours.

In a twenty sixth embodiment according to the seventeenth through twenty fourth embodiments above, the supercritical fluid extraction process is carried out within a time period of from 2 to 15 hours.

In a twenty seventh embodiment according to the seventeenth through twenty fourth embodiments above, the supercritical fluid extraction process is carried out within a time period of from 3 to 9 hours.

In a twenty eighth embodiment of the present invention, a method for decannabinization of marijuana is provided comprising subjecting marijuana plants to supercritical fluid extraction to remove delta-9-THC and related cannabinoids therefrom, wherein the delta-9-THC concentration of said marijuana is from about 0-0.5 wt. % after supercritical fluid extraction thereof.

In a twenty ninth embodiment of the present invention according to the twenty eighth embodiment above, the supercritical fluid extraction process for decannabinization of marijuana is carried out within a temperature range of from 25 to 65° C.

In a thirtieth embodiment of the present invention according to the twenty ninth embodiment above, the supercritical fluid extraction process is carried out at a pressure of from 400-500 bar.

In a thirty first embodiment of the present invention according to the twenty eighth embodiment above, a placebo marijuana cigarette is provided comprising the decannabinized marijuana produced by the process of the twenty eighth through thirtieth embodiments above, wherein the placebo marijuana cigarette has a concentration of Delta-9-THC and related cannabinoids of about 0 to about 0.5 wt %.

In a thirty second embodiment of the present invention according to the first through thirtieth embodiments above, the supercritical fluid extraction process is carried out at a flow rate of 20-50 ml/minute per 80 g of marijuana plant.

In a thirty third embodiment of the present invention according to the second through twenty seventh embodiments of the present invention, the organic cosolvent modifier comprises from greater than 0 to about 20 wt % of the supercritical fluid, based on the total amount of supercritical fluid.

Carbon dioxide is most preferred for the extraction process since it is nonflammable, nontoxic, less expensive than reagent grade liquid solvents, available in a high state of purity, and can be vented to the atmosphere or recycled without harm to the environment. Moreover, the SCF-CO₂ extractions can be performed under relatively mild conditions, thus, reducing the risks of thermal degradation and poor collection efficiencies of volatile analytes. Currently, CO₂ is recognized as safe, and is regulated by the U.S. Food and Drug Administration [CFR 21.184.1240 (C)] as a direct human food ingredient.

In the most preferred embodiment, the liquid CO₂ is used in a purified form, i.e., having a purity of from 95 to 100 wt % of CO₂. Suitable cosolvents, which may be used in combination with the liquid CO₂ include ethanol, methanol, 2-propanol, ethyl acetate, acetonitrile, carbon tetrachloride, hexane, cyclohexane, and other nonpolar and semipolar solvents in an amount of from about 0 to 20 wt % of the total wt. of liquid supercritical fluid being used.

In a preferred embodiment, liquid CO₂ is used in the supercritical extraction process of Delta-9-Tetrahydrocannabinol and Delta-8-Tetrahydrocannabinol and other cannabinoids at temperatures ranging from 25° C. to 65° C., more preferably from 30 to 65° C., most preferably from 35 to 45° C. The supercritical extraction process is carried out at pressures ranging from about 70 to 550 bar, more preferably from 100 to 500 bar and most preferably from 400 to 500 bar.

According to the present invention, marijuana plant material is maintained in contact with the supercritical liquid CO₂ under the above temperature and pressure conditions for a period of from about 0 to 24 hours, preferably from about 2 to about 15 hours, more preferably from about 3 to about 9 hours, so as to facilitate the desired amount of removal of the cannabinoids from the marijuana plant material.

### DETAILED DESCRIPTION OF THE INVENTION

The present invention provides a method of extraction of pharmaceutically active compounds from natural resources (such as marijuana plants) and use of the extracted compounds, either pure or mixture, for pharmaceutical dosage forms. The processed matrix (marijuana subjected to supercritical fluid extraction of the present invention) with defined strengths of active(s) ingredients, single compound or mixture of compounds, can be used for making delivery devices, such as marijuana cigarettes, with specific, known concentrations of Delta-9-THC and related pharmaceutically active compounds.

The extraction process of the present invention is carried out using supercritical fluid, preferably liquid CO₂, either alone or in combination with other cosolvents, so as to retain the natural properties of the marijuana plant material.

In the most preferred embodiment, the liquid CO₂ is used in a purified form, i.e., having a purity of from 95 to 100 wt % of CO₂. Suitable organic cosolvent modifiers, which may be used in combination with the liquid CO₂ include ethanol, methanol, 2-propanol, ethyl acetate, acetonitrile, carbon tetrachloride, hexane, cyclohexane, and other nonpolar and semipolar solvents. These cosolvents are present in an amount of from about 0 to about 20 wt % of the total wt. of liquid supercritical fluid being used. In certain instances, the use of a cosolvent may be advantageous for the purpose of selectivity, ease of extraction and shorter required extraction times. However, where volatile organic cosolvents are used, this may present an environmental problem and additional expense in insuring there is little or no escape of the organic cosolvents into the atmosphere.

In a preferred embodiment, liquid CO₂ is used in the supercritical extraction process of Delta-9-Tetrahydrocannabinol and Delta-8-Tetrahydrocannabinol and other cannibinoids at temperatures ranging from 25° C. to 65° C., more preferably from 30 to 65° C., most preferably from 35 to 45° C. The supercritical extraction process is carried out at pressures ranging from about 70 to 680 bar, more preferably from 100 to 500 bar and most preferably from 400 to 500 bar.

According to the present invention, marijuana plant material is maintained in contact with the supercritical liquid CO₂ under the above temperature and pressure conditions for a period of from about 0 to 24 hours, preferably from about 2 to about 15 hours, more preferably from about 3 to about 9 hours, so as to facilitate the desired amount of removal of the cannabinoids from the marijuana plant material.

The addition of an organic cosolvent modifier, as called for in the eighth embodiment, serves to increase fluid polarity, rather than alternate fluids such as propane, butane, and isobutane. However, it is within the scope of the invention to employ preferred supercritical fluids of carbon dioxide, carbon monoxide, water, ethane, ammonia, nitrous oxide, fluoroform, and xenon for the extraction of cannabinoids from marijuana plant materials.

In a preferred embodiment, the supercritical extraction process using liquid CO₂ is carried out in combination with a cosolvent comprising one or more of ethanol, methanol, 2-propanol, ethyl acetate, acetonitrile, carbon tetrachloride, hexane, cyclohexane, and other nonpolar and semipolar solvents, wherein the cosolvent can constitute from about 0 to 20 wt % of the total supercritical fluid used in the extraction. When a cosolvent of nonpolar and semipolar in nature is used with liquid CO₂, the process is preferably carried out at a temperature range from about 30° C. to about 40° C., and at a pressure of from about 100 bar to about 400 bar, for a period of from about 3 to 7 hours.

During the extraction of cannabinoids from marijuana plant material, it is preferred that the supercritical fluid pass into contact with the plant material at a flow rate of from about 20 to 50 ml/min based on 80 gms of marijuana plant material being processed.

To increase the rate of extraction of the cannabinoids from the marijuana plant material, the flow rate of the supercritical fluid can be increased, as well as the residence time of the marijuana plant material in contact with the supercritical fluid.

The preferred solvent, liquid carbon dioxide, used in the SCFE process is environmentally safe and does not leave any residues. A small proportion of organic cosolvents addition for decannabinization of marijuana by SCFE will also remove the active ingredients under these mild operating conditions. Different process conditions may yield marijuana extracts with different amounts of THC.

Low density SF—CO₂ has the polarity of hexane. However, SF polarity increases with density, especially near the critical point. At its highest density, SF—CO₂ resembles the polarity of solvents such as toluene, benzene, and ether. In the supercritical state, CO₂ is at its critical temperature (31.3° C.) and is in its gaseous phase under high pressure (70-1500 bar).

According to the present invention, decannabinization of marijuana plant parts can be achieved under relatively mild conditions, and the processed marijuana unexpectedly retains its appearance/color and texture, irrespective of process conditions. The repeatability of this extraction process has also been demonstrated to remove delta-9-tetrahydrocannabinol present in the marijuana to a content of ˜0-0.5 wt. % starting from as high as 3.4 wt. %.

In another embodiment, cigarette machines can be easily modified to suit the handling of marijuana plant parts to produce marijuana cigarettes similar to commercial grade (e.g. tobacco) cigarettes. Placebo cigarettes can also be produced using SCFE treated marijuana. Applicants have successfully scaled up the SCFE process for amounts of marijuana from ˜25 g to ˜80 g.

Smoking of both untreated marijuana cigarettes and SCFE treated marijuana cigarettes were carried out successfully to determine the THC delivered from such device. Condensates taken from the cigarettes tested were analyzed by gas chromatography. Spiking of the placebo cigarettes with standardized THC content can be used to produce cigarettes having different strengths. Thus, according to the present invention, titrated cigarettes of different strengths can be produced which are excellent for clinical studies. The present invention leads to ready availability of an alternate natural source of THC to the synthetic sources. The selectively extracted compounds or mixture of compounds can be administered through different delivery devices for treatment of patients with severe ailments.

### TEST EXAMPLES

### Notes for all Tables

Concentration refers to the amount of delta-9-THC present in a unit volume of the analytical sample(s) prepared from either untreated or SCF treated marijuana or the SCF marijuana extract. For instance, C-00-001 is virgin marijuana, which was analyzed to estimate the amount (% w/w) of delta-9-THC present in marijuana. The concentration was measured by Gas Chromatography (GC). THC can also be analyzed by HPLC and other analytical techniques. The remaining marijuana samples are SCF-treated and extracts obtained therefrom.

% Delta-9-THC, refers to the amount of delta-9-THC present in a particular material, i.e. either the virgin marijuana or SCF-treated marijuana or SCF marijuana extract. These values are calculated based on the concentration observed in the analytical samples. Avg. % of delta-9-THC, is the sum of all (%) values of delta-9-THC divided by the total number of samples analyzed from a particular material.

### Example 1

A small quantity (25 g) of marijuana plant material was obtained, so as to subject same to supercritical fluid (SCF) extraction, and started with random extraction conditions.

Initially, two samples of a quantity of virgin (natural, unextracted) marijuana (designated C-00-001 in Tables 1-3) was analyzed using gas chromatography to determine the amount of delta-9-THC therein. As shown in Table 1, it was determined that Lot # C-00-001 of virgin marijuana contained an average of 2.76% delta-9-THC, based on weight.

Then, a first sample of the virgin marijuana was subjected to SCF extraction under 150 bar pressure, with a flow rate of liquid CO₂ of 20 g/min, at 58° C. bath temperature, for a period of 4 hours, and a first analytical sample was obtained. A second sample of the virgin marijuana was subjected to SCF extraction as above, and a second analytical sample obtained. The first and second analytical samples were then analyzed using gas chromatography (GC) to determine the concentration of delta-9-THC present in the marijuana after extraction of delta-9-THC therefrom using the process above. The results of these GC measurements are shown in Table 1 below, labeled as “C-00-00”, which show that the virgin marijuana has an avg. % delta-9-THC of 1.88%.

The extracts obtained from the supercritical fluid extraction of the first and second samples above were then analyzed using GC analysis, to determine the amount of delta-9-THC present in the extracts. The results of these analyses are shown in Table 1. As shown below, approximately 30% of the total amount of delta-9-THC present in the sample was removed from the plant material, i.e., only about one-third of the delta-9-THC was extracted from the plant parts. This is reflected in the extract analysis (shown in Table 1).

TABLE 1
Delta-9-THC (Analysis (Lot #001101)

| | Sample | Concentration | % Delta-9- | Avg. % Delta- |
|---|---|---|---|---|
| Lot # | Weight (mg) | (ug/mL) | THC | 9-THC |
| C-00-001 | 97.4 | 920.90 | 2.84 | 2.76 |
| | 100.1 | 895.57 | 2.68 | |
| 001101 | 101.1 | 597.86 | 1.77 | 1.88 |
| SCFE | 98.9 | 655.3 | 1.99 | |
| Marijuana | | | | |
| 001101 | 23.0 | 840.70 | 36.55 | 36.74 |
| Extract | 17.2 | 635.11 | 36.93 | |

### Example 2

A first sample of marijuana was taken from Lot # C-00-001, consisting of a marijuana leaf (designated “M. Leaf” in Table 2 below), and a second sample was taken from Lot # C-00-001, consisting of crushed marijuana leaves (designated “M. Leaf Crushed”).

Each of said samples above was analyzed using GC analysis, and the concentration in wt. % of delta-9-THC in the samples was determined. The results of these analyses is shown in Table 2 below (designated as “C-00-001, M. Leaf” and “C-00-001 M. Leaf Crushed”, respectively), where it can be seen that the virgin marijuana has a concentration of as much as 3.4 wt. %.

Then, two samples of the extracted marijuana plant material from the first and second analytical samples obtained above (from Lot # 001101 shown above) was then prepared, the first sample consisting of marijuana leaves (designated “M. Leaf” in Table 2) and the second sample consisting of crushed marijuana (designated “Marijuana Crushed” in Table 2). Each of these samples was then re-extracted with liquid CO₂ under a pressure of 400 bar at a 50 g/min flow rate for a period of 5 hours to obtain a third and fourth analytical sample (designated as “001102, SCFE Marijuana (M. Leaf)” and “001102, SCFE Marijuana Crushed”, respectively).

After re-extraction of the third and fourth samples, as described above, GC analysis was carried out to determine the concentration of delta-9-THC present in the sample. As shown in Table 2 below, this process reduced the delta-9-THC in the sample from 3.11 wt. % to 0.15 wt. %, and 0.21 wt. % from 3.39 wt. %, respectively.

In addition, GC analysis was carried out on each of the extracts (both designated as “001102, M. SCFE Extract) obtained in the re-extraction of the third and fourth samples above. The results of these analyses are shown in Table 2 below:

TABLE 2
Delta-9-THC Analysis (Lot #001102)

| | Sample | Concentration | % Delta-9- | Avg. % Delta- |
|---|---|---|---|---|
| Lot # | Weight (mg) | (ug/mL) | THC | 9-THC |
| 001102, | 95.1 | 46.88 | 0.15 | 0.15 |
| SCFE | | | | |
| Marijuana | | | | |
| (M. leaf) | | | | |
| 001102, | 92.6 | 66.26 | 0.21 | 0.21 |
| SCFE | | | | |
| Marijuana | | | | |
| Crushed | | | | |
| 001102, | 18.1 | 788.32 | 43.55 | 47.22 |
| M. SCFE | 18.3 | 931.26 | 50.89 | |
| Extract | | | | |
| C-00-001, | 102.7 | 1099.30 | 3.11 | 3.11 |
| M. Leaf | | | | |
| C-00-001 | 97.2 | 1135.87 | 3.39 | 3.39 |
| M. Leaf | | | | |
| Crushed | | | | |

### Example 3

Another batch (Lot #001104) of 25 g of marijuana was obtained, 2 samples taken therefrom, and the samples subjected to supercritical fluid extraction having the following conditions: 30 g/min flow rate with liquid CO2 under 450 bar at 62° C. bath temperature for 6 hours. Then, these two samples (both designated as “SCFE Marijuana” in Table 3 below) were subjected to GC analysis to determine the concentration of delta-9-THC therein, the results of these analyses shown in Table 3 below. As shown, the above SCF extraction conditions resulted in an improved reduction of delta-9-THC concentration of from 3.4% to 0.1%.

TABLE 3
Delta-9-THC Analysis (Lot #001104)

| | Sample | Concentration | % Delta-9- | Avg. % Delta- |
|---|---|---|---|---|
| Sample | Weight (mg) | (ug/mL) | THC | 9-THC |
| SCFE | 95.8 | 38.10 | 0.12 | 0.12 |
| Marijuana | 94.6 | 38.05 | 0.12 | |
| SCFE M. | 17.3 | 668.75 | 38.66 | 38.24 |
| Extract | 26.7 | 1009.68 | 37.82 | |

### Example 4

Another batch of virgin marijuana, containing about 25 g of marijuana, was obtained, and 6 samples prepared therefrom (designated as “Sample Number” 1-6 in Table 4). Each of these samples was then processed by SCFE under different pressures, temperature, and time. (100 bar, 32 C., 20 g/min flow for 1 hour; 200 bar with the same conditions as before; 300 bar under the same conditions as before; 400 bar same conditions as before; 500 bar for 5 hours at 59 C. bath temperature) temperatures and time conditions.

This attempt lead to the reduction of delta-9-THC to 0.49%. The delta-9-THC present in the individual extracts varied from ˜20% to ˜46%.

### Example 5

Another batch of 25 g of marijuana (designated as Lot # 001201R&D) was obtained, several samples prepared therefrom, and the samples processed using SCF extraction under different conditions of pressures of 72 (I set), 400 (II set), and 400(III set) bar, at a temperature of 31 (I), 31(II), and 60(III) ° C. for a period of 3.5 (I), 4.0 (II), and 2.5 (III) hours under a flow rate of 20 (I), 20 (II) and 30(III) g/min, respectively. The processed samples were then analyzed using GC analysis, the results thereof confirming that the process of decannabinization was repeatable, and the extent of delta-9-THC reduction depends upon the processing conditions.

The data for the lot #001201R&D is presented in Table 4 below. Different SCFE extracts obtained from marijuana utilizing different processing conditions exhibited a ˜20 to ˜46% reduction of delta-9-THC concentration. All these samples were analyzed by Gas chromatography.

TABLE 4
Delta -9- THC Analysis (Lot # 001201R&D)

| | | Sample | | | |
|---|---|---|---|---|---|
| | Sample | Weight | Concentration | % | Avg. % Delta- |
| Sample | Number | (mg) | (ug/mL) | Delta-9-THC | 9-THC |
| Marijuana | 1 | 95.5 | 75.33 | 0.24 | 0.23 |
| | 2 | 96.1 | 71.55 | 0.22 | |
| | 3 | 104.5 | 81.49 | 0.23 | |
| | 4 | 101.4 | 74.33 | 0.22 | |
| | 5 | 94.3 | 59.32 | 0.19 | |
| | 6 | 102.6 | 87.09 | 0.25 | |
| Extract I | A | 22.5 | 952.59 | 42.34 | 41.73 |
| Powder | B | 16.5 | 678.65 | 41.13 | |
| Extract I | A | 18.2 | 613.48 | 33.71 | 33.36 |
| Sticky | B | 20.7 | 683.46 | 33.02 | |
| Extract III | A | 20.9 | 598.61 | 28.64 | 28.62 |
| | B | 22.9 | 654.72 | 28.59 | |

### SCALE UP OF SCF EXTRACTION

### Example 6

A scaled up batch of ˜80 g of marijuana obtained from Lot #R010101, samples prepared therefrom and said samples processed using SCF under different conditions of a pressures of 72 (I set), 400 (II set), and 400 (III set) bar, at a temperature of 31 (I), 31 (II), and 51 (III) ° C. for a period of 0.5 (I), 4.0 (II), and 5 (III) hours under a flow rate of 20 (I), 20 (II) and 30(III) g/min, respectively. The scale up SCF extraction process was conducted to establish the extraction efficiency on scale up batch levels.

Each of the samples was then subjected to GC analysis to determine the delta-9-THC concentration in the samples after SCF extraction of delta-p-THC. The results confirmed that the process of decannabinization is repeatable, and the extent of delta-9-THC reduction depends on the processing conditions. Data concerning each of the samples prepared from lot #R010101 is presented in Table 5 below. Different SCFE extracts obtained from the marijuana utilizing different processing conditions exhibited about 8 to 40% reduction of delta-9-THC in the marijuana. All of these samples were analyzed by gas chromatography.

TABLE 5
Delta -9- THC Analysis (Lot # R010101)

| | Sample | Sample | Concentration | % | Avg. % Delta- |
|---|---|---|---|---|---|
| Sample | Number | Weight (mg) | (ug/mL) | Delta-9-THC | 9-THC |
| Marijuana | 1 | 109.1 | 82.61 | 0.23 | 0.23 |
| | 2 | 97.1 | 77.42 | 0.24 | |
| | 3 | 96.7 | 74.40 | 0.23 | |
| | 4 | 103.4 | 76.06 | 0.22 | |
| | 5 | 96.3 | 70.48 | 0.22 | |
| | 6 | 97.0 | 69.41 | 0.21 | |
| Extract 1 | A | 94.8 | 162.60 | 8.58 | 8.58 |
| Extract 2 | A | 121.8 | 952.73 | 39.11 | 40.07 |
| | B | 102.7 | 842.86 | 41.04 | |
| Extract 3 | A | 102.0 | 653.42 | 32.03 | 32.49 |
| | B | 111.7 | 735.9 | 32.94 | |

### Example 7

Another scale up batch of about 80 g of marijuana was obtained (designated as Lot # R010200), samples prepared therefrom, and said samples subjected to SCF extraction under conditions similar to those in Example 6, with minor variations of a pressure of 72 (I set), 400 (II set), and 450 (III set) bar, at a temperature of 31 (I), 31 (II), and 45 (III) ° C. for a period of 1.0 (I), 4.0 (II), and 5 (III) hours under a flow rate of 20 (I), 20 (II) and 30(III) g/min, respectively. Each of the samples was then subjected to GC analysis, to determine the concentration of delta-9-THC therein after SCF extraction was carried out thereon. Test data for each of said samples is presented in Table 6 below. The data demonstrates that the process of the present invention is repeatable, but the efficiency did not improve further.

TABLE 5
Delta -9- THC Analysis (Lot # R010200)

| | Sample | Sample | Concentration | % | Avg. % Delta- |
|---|---|---|---|---|---|
| Sample | Number | Weight (mg) | (ug/mL) | Delta-9-THC | 9-THC |
| Marijuana | 1 | 104.8 | 104.66 | 0.30 | 0.30 |
| | 2 | 106.2 | 119.16 | 0.34 | |
| | 3 | 97.8 | 88.18 | 0.27 | |
| | 4 | 96.2 | 94.86 | 0.30 | |
| | 5 | 97.4 | 100.77 | 0.31 | |
| | 6 | 108.3 | 111.32 | 0.31 | |
| Extract 1 | A | 25.2 | 508.22 | 20.17 | 19.85 |
| | B | 23.8 | 464.64 | 19.52 | |
| Extract 2 | A | 21.6 | 743.85 | 34.44 | 37.58 |
| | B | 19.4 | 789.86 | 40.71 | |
| Extract 3 | A | 22.0 | 614.15 | 27.92 | 28.50 |
| | B | 15.5 | 450.95 | 29.09 | |

### Example 8

The scaled up batch process illustrated in Example 7 above was repeated. However, the SCF extraction was carried out at an increased temperature of from 45 to 60° C. (in IIIrd set only). It was unexpectedly discovered that increasing the temperature to within this narrow range improved the extraction results, i.e., more delta-9-THC was removed from the sample, resulting in near complete removal of delta-9-THC from the marijuana plant parts. This data is illustrated in Table 7 below.

TABLE 7
Delta -9- THC Analysis (Lot # R010201)

| | Sample | Sample | Concentration | % | Avg. % Delta- |
|---|---|---|---|---|---|
| Sample | Number | Weight (mg) | (ug/mL) | Delta-9-THC | 9-THC |
| Marijuana | 1 | 98.9 | 47.44 | 0.14 | 0.14 |
| | 2 | 105.9 | 43.58 | 0.12 | |
| | 3 | 101.1 | 48.13 | 0.14 | |
| | 4 | 102.3 | 42.83 | 0.13 | |
| | 5 | 103.2 | 49.81 | 0.14 | |
| | 6 | 105.2 | 49.08 | 0.14 | |
| Extract 1 | A | 24.4 | 407.46 | 16.70 | 17.38 |
| | B | 18.8 | 339.73 | 18.07 | |
| Extract 2 | A | 26.2 | 922.53 | 35.21 | 36.52 |
| | B | 20.8 | 787.00 | 37.84 | |
| Extract 3 | A | 20.9 | 348.76 | 16.69 | 15.87 |
| | B | 24.3 | 365.56 | 15.04 | |

### Example 9

Another scale up batch of marijuana was obtained (designated as Lot # R010201), samples prepared therefrom, and the SCF extraction process described in Example 8 repeated on large scale, with the elimination of one (I set) set of conditions. More specifically, the samples were processed under two sets of conditions, i.e., of pressures of 400 (I set) and 450 (II set) bar, at temperatures of 34 (I) and 50 (II) ° C., and for periods of 4.0 (I) and 7.0 (II) hours under flow rates of 20 (I) and 30(II) g/min, respectively. Each of the samples was then analyzed using gas chromatography, to determine the concentration of the delta-9-THC present in each of the samples after SCF extraction was carried out theron.

The results of these tests are presented in Table 8 below. These test results show that the SCF extraction of marijuana plant parts is feasible, and the marijuana retains its original color for making placebo cigarettes, or for spiking placebo cigarettes.

TABLE 8
Delta -9- THC Analysis (Lot # R010202)

| | Sample | Sample | Concentration | % | Avg. % Delta- |
|---|---|---|---|---|---|
| Sample | Number | Weight (mg) | (ug/mL) | Delta-9-THC | 9-THC |
| Marijuana | 1 | 99.7 | 48.56 | 0.15 | 0.14 |
| | 2 | 103.4 | 48.46 | 0.14 | |
| | 3 | 110.7 | 46.39 | 0.13 | |
| | 4 | 104.4 | 52.78 | 0.15 | |
| | 5 | 103.3 | 48.87 | 0.14 | |
| | 6 | 99.2 | 51.75 | 0.16 | |
| Extract 1 | A | 22.6 | 686.77 | 30.39 | 34.18 |
| | B | 20.0 | 759.52 | 37.98 | |
| Extract 2 | A | 23.8 | 508.86 | 21.38 | 20.99 |
| | B | 22.8 | 469.45 | 20.59 | |

### Example 10

Another batch of virgin marijuana was obtained (designated as Lot # R020408, consisting of about 25 g of marijuana), four samples prepared therefrom, and said samples processed by SCF extraction under the following conditions: a pressure of 450 bar, a temperature of 55° C., and a flow rate of 30 g liquid carbon dioxide/min for 7 hours. The processed material (i.e., marijuana subjected to SCF extraction) was then re-extracted with differing amounts of ethanol (10, 20, 30 and 40 ml ethanol, respectively) under a pressure 350 bar, a temperature of 50° C., and a flow rate of 30 g of liquid carbon dioxide/min at four different times for one hour each. As mentioned above, the amount of ethanol used was 10, 20, 30 to 40 ml for the four different process cycles, respectively.

The four SCFE marijuana samples were then analyzed by GC. The results showed that the Delta-9-THC in marijuana was removed to the extent of 0.07%. i.e., the re-extracted samples were “decannabinized marijuana” as desired for use in placebo marijuana cigarettes.

As mentioned above, for the analysis of delta-9-THC extracted from marijuana plant parts and SCF extracts, gas chromatography was used in all cases. Marijuana plant extraction was carried out with organic solvent systems for sample analysis. In the analysis ˜20-100 mg of marijuana, or its extract, was introduced into a test tube. 10 mL of an extraction solvent (90:10 methanol:chloroform) containing 1000 g/mL of internal standard (4-androstene-3,17-dione) was then added to the marijuana or marijuana extract. The solution was then sonicated for ˜10 minutes to break up the lumps, and centrifuged to separate the suspension from the supernatant. The supernatant was then subjected to GC analysis.

### MARIJUANA CIGARETTE PREPARATION

### Example 11

A first blend of about 300 g of untreated marijuana plant parts was humidified to raise the moisture content of the marijuana by sprinkling water and leaving the marijuana overnight to absorb the water and produce humidified marijuana. The humidified marijuana was then used for rolling cigarettes using a cigarette machine, which was modified to suit the handling of marijuana plant parts. The process resulted in a high quality of marijuana cigarettes suitable for smoking experiments.

Similarly, a second blend of SCF-extracted marijuana (i.e., so-called “decannabinized marijuana” having a low delta-9-THC concentration) pooled from different batches of extraction formed one blend was produced. This blend was utilized to make marijuana cigarettes upon humidification. A good quality of cigarettes was obtained from the SCF processed marijuana. The quality of cigarettes was verified by the Quality Control Department of Murty Pharmaceuticals, Inc. (MPI).

### FTC SMOKE TESTING OF CIGARETTES

### Example 12

The marijuana cigarettes made from both untreated and SCF treated marijuana were used for initial smoke testing (FTC). It was found that the cigarettes made at MPI were of high quality in terms of handling, testing, and appearance. Importantly, the THC content present in the placebo cigarettes produced by MPI was negligible. These placebo cigarettes are important for use as a control in marijuana smoke testing.

As demonstrated by the test results shown in the above Examples, SCF extraction of marijuana plant parts for the removal of THC proved to be feasible and repeatable under mild and environmentally acceptable conditions. Selective extraction was achieved, by varying the processing conditions, such as pressure, temperature and duration of the extraction process.

What is claimed is:

1. A process for the extraction of Delta-9-THC and other related pharmaceutically active compounds from marijuana plants, said process comprising supercritical fluid extraction of Delta-9-THC, Delta-8-THC and related cannabinoids from marijuana plants using liquid carbon dioxide as a supercritical fluid.

2. The process of claim 2, further comprising use of an organic cosolvent modifier in the extraction of Delta-9-THC, Delta-8-THC and other related pharmaceutically active compounds therein, from marijuana plants.

3. The process of claim 1, wherein the supercritical fluids used in the supercritical fluid extraction process are one or more selected from the group comprising NH₃, N₂O, ethanol, pentane, and propane, and high purity carbon dioxides.

4. The process of claim 1, wherein the supercritical fluid extraction using CO₂ is carried out at or above its critical temperature of 31.3° C. and at a pressure of 70 bar.

5. The process of claim 1, wherein the super critical fluid extraction process is carried out within an operating temperature range of from 31 to 120 ° C.

6. The process of claim 1, wherein the supercritical fluid extraction process is carried out within an operating temperature range of from about 25-65° C.

7. The process of claim 1, wherein the supercritical fluid extraction process is carried out within an operating temperature range of from about 30-65° C.

8. The process of claim 1, wherein the supercritical fluid extraction process is carried out within an operating temperature range of from about 35-45° C.

9. The process of claim 1, wherein the supercritical fluid extraction process is carried out within a pressure range of from about 70 to about 680 bar.

10. The process of claim 1, wherein the supercritical fluid extraction process is carried out within a preferred pressure range of from about 100 to about 500 bar.

11. The process of claim 1, wherein the supercritical fluid extraction process is carried out within a preferred pressure range of from about 400 to about 500 bar.

12. The process of claim 1, wherein the supercritical fluid extraction process is carried out within a time period of from 0 to 24 hours.

13. The process of claim 1, wherein the supercritical fluid extraction process is carried out within a time period of from 2 to 15 hours.

14. The process of claim 1, wherein the supercritical fluid extraction process is carried out within a time period of from 3 to 9 hours.

15. The process of claim 1, wherein the super critical fluid extraction process is carried out using a combination of carbon dioxide and an organic cosolvent modifier.

16. The process of claim 16, wherein the organic cosolvent modifier is one or more selected from the group consisting of ethanol, methanol, 2-propanol, diethylether, ethyl acetate, chloroform, carbontetrachloride, acetonitrile, cyclohexane, acetone, acetic acid, nitromethane, dioxane, methylene chloride, hexane, pentane, acetylene, and pyridine.

17. A process for extracting Delta-9-THC, Delta-8-THC and related pharmaceutically active compounds comprising supercritical fluid extraction of marijuana, wherein the supercritical fluids used in said process comprise one or more selected from the group consisting of carbon dioxide, carbon monoxide, water, ethane, ammonia, nitrous oxide, fluoroform, and xenon.

18. The process of claim 17, wherein the super critical fluid extraction process is carried out within an operating temperature range of from 31 to 120 ° C.

19. The process of claim 17, wherein the supercritical fluid extraction process is carried out within an operating temperature range of from about 25-65° C.

20. The process of claim 17, wherein the supercritical fluid extraction process is carried out within an operating temperature range of from about 30-65° C.

21. The process of claim 17, wherein the supercritical fluid extraction process is carried out within an operating temperature range of from about 35-45° C.

22. The process of claim 17, wherein the supercritical fluid extraction process is carried out within a pressure range of from about 70 to about 680 bar.

23. The process of claim 17, wherein the supercritical fluid extraction process is carried out within a preferred pressure range of from about 100 to about 500 bar.

24. The process of claim 17, wherein the supercritical fluid extraction process is carried out within a preferred pressure range of from about 400 to about 500 bar.

25. The process of claim 17, wherein the supercritical fluid extraction process is carried out within a time period of from 0 to 24 hours.

26. The process of claim 17, wherein the supercritical fluid extraction process is carried out within a time period of from 2 to 15 hours.

27. The process of claim 17, wherein the supercritical fluid extraction process is carried out within a time period of from 3 to 9 hours.

28. A method for producing decannabinized marijuana comprising subjecting marijuana plants to a supercritical fluid extraction process to remove delta-9-THC and related cannabinoids therefrom, wherein the delta-9-THC concentration of said decannabinized marijuana is from about 0-0.5 wt. % after supercritical fluid extraction thereof.

29. The method of claim 28, wherein the supercritical fluid extraction process for decannabinization of marijuana is carried out within a temperature range of from 25 to 65° C.

30. The method of claim 29, wherein the supercritical fluid extraction process is carried out within a pressure range of from 400-500 bar.

31. A placebo marijuana cigarette comprising the decannabinized marijuana produced by the method of claim 28, wherein the placebo marijuana cigarette has a concentration of Delta-9-THC and related cannabinoids of about 0 to about 0.5 wt %.

32. The process of claim 1, wherein the supercritical fluid extraction process is carried out at a flow rate of 20-50 ml/minute per 80 g of marijuana plant.

33. The process of claim 2, wherein the organic cosolvent modifier comprises from greater than 0 to about 20 wt % of the supercritical fluid, based on the total amount of supercritical fluid.
